(19) United States
(12) Patent Application Publication
(10) Pub. No.: US 2010/0129295 A1
(43) Pub. Date: May 27, 2010

(54) Compositions for preventing and/or treating gingivitis

(76) Inventors: Jiajiu Shaw, Ann Arbor, MI (US); Jen Chuan Wang, Hacienda Heights, CA (US); Michael S. Gzybowski, Ann Arbor, MI (US)

Correspondence Address: Michael S. Gzybowski, 832 Earhart Road, Ann Arbor, MI, 48105, US

(21) Appl. No.: 12/313,678

(22) Filed: Nov. 22, 2008

(51) Int. Cl. A61K 8/22; A61K 8/30; A61K 9/68; A61Q 11/00

(52) U.S. Cl. 424/48; 424/49; 424/53

(57) ABSTRACT

Compositions and methods for preventing and/or treating gingivitis which include the use of one or a plurality of TNF-α modulators.

### TECHNICAL FIELD

Compositions for prevention and/or treatment of gingivitis

### BACKGROUND AND PRIOR ART

Gingiva (gums) are part of the soft tissue lining in the mouth surrounding the teeth. Gingivitis is an inflammation of the gums that often appears as swollen, red, or bleeding gums. It is generally believed that plaque-forming bacteria that live in the mouth and on tooth surfaces are a cause of gingivitis and certain substances released by the bacteria cause the gum inflammation. Therefore, reducing plaque bacteria can decrease inflammatory substances and effect a reduction in gingivitis.

Current preventative measures for reducing gingivitis include good oral hygiene such as the use of antiplaque or antitartar toothpastes and the use of mouth rinses and floss. These products loosen and remove deposits/bacteria from the teeth and clean the teeth.

For treatment of gingivitis antibacterial mouthwash can be used in addition to frequent, careful, tooth brushing and flossing. This type of mouthwash typically comprises hexetidine, chlorhexidine digluconate, or cetylpyridinium chloride. The goal for this type of product is to reduce the amount or effects of microorganisms and bacteria.

Current preventive measurements and treatment protocol are not satisfactory and a significant improvement is needed. The common problem of all current preventative measurements and treatment protocol is that neither directly address or treat inflammation, including the inflammatory stimuli secreted by bacteria, which is the root cause of inflammation. Once the inflammatory stimuli contact the gums, the body's immune system will produce a number of responses including upregulation of cytokines such as tumor necrosis factor alpha (TNF-α). None of the current preventative measurements and treatment protocol are directed at controlling the immune responses including upregulation of TNF-α.

### Role of TNF-A in Gingivitis

Among a number of cytokines related to inflammation, TNF-α is probably the most critical cytokines. Elevated concentrations of plasma TNF-α have been found in a variety of infections and inflammatory diseases indicating the important role of TNF-α in the immune-mediated diseases such as rheumatoid arthritis (RA). Therefore, modulation of TNF-α is the focus of research in the development of immune-mediated diseases.

According to Nilsson et al. (Journal of Periodontology Online 2008; 79: 1689-1696), gingivitis and periodontitis are related to repeated high levels of circulating TNF-α in patients with RA.

All current gingivitis products are designed to remove or kill bacteria. In fact, the inflammation associated with gingivitis is caused directly by the stimuli secreted by bacteria. However, once the stimuli contact the gums, removing bacteria is not enough. Surprisingly, there is no gingivitis product that has been developed to modulate the inflammatory response by the immune system, not to mention lowering the abnormally elevated TNF-α levels.

Bacteria around the gums and stimuli secreated by the bacteria cause the upregulation of TNF-α. Administration of TNF-α modulator(s) can prevent the elevated TNF-α levels and/or maintain normal TNF-α levels. When the elevated TNF-α is lowered to around normal levels, the inflammation of the gums will be significantly reduced. Assuma et al. (The Journal of Immunology 1998; 160: 403-409) has shown that injection of soluble receptors of IL-1 and TNF-α to sites with induced periodontal destruction inhibits the about 80% the recruitment of inflammatory cells. Assuma et al. showed that IL-1 and TNF-α antagonists inhibit the inflammatory response and bone loss in experimental periodontitis. They applied soluble receptors of IL-1 and TNF-α to patients by local injection to sites with induced periodontal destruction and compared with similar sites injected with vehicle alone. The results indicate that injection of soluble receptors to IL-1 and TNF-α inhibited by approximately 80% the recruitment of inflammatory cells in close proximity to bone.

U.S. Pat. No. 6,727,272 to Lee et al. discloses that a number of small-molecule TNF-α modulators are anti-inflammatory.

Other significant ingredients that are shown to be effective against gingivitis include vitamin B2 and calcium. Petti et al. (Community Dentistry & Oral Epidemiology. 28(6):407-13, December 2000) conducted a study looking at various nutritional factors that are associated with gingivitis. A total of 27 females with gingivitis were compared to women of the same age and risk factors who did not have gingivitis. Those females who took supplements of calcium and vitamin B2 had reduced risk of gingivitis indicating that vitamin B2 and calcium protect against gingivitis.

### DISCLOSURE OF THE INVENTION

The present invention provides a composition for preventing and/or treating gingivitis wherein the composition comprises one or a plurality of naturally occurring TNF-α modulators.

The present invention further provides a composition for preventing or treating gingivitis and/or maintaining healthy gums wherein the composition comprises one or a plurality of synthetic TNF-α modulators.

The present invention also provides methods for preventing or treating gingivitis and/or maintaining healthy gums comprising orally administrating the composition. Such oral administration can include having a subject use a form of the composition as liquid mouthwash or rinse, in a toothpaste composition, as a chewing gum, or a lozenge, or any manner that exposes the subject's gums to the compositions.

### BEST MODE FOR CARRYING OUT THE INVENTION

The present invention is directed to a composition for preventing and/or treating gingivitis which comprises one or a plurality of naturally occurring TNF-α modulators.

The composition disclosed herein is unique and different from all currently known products for treating gingivitis. The basic principle of the present invention is to lower the abnormal levels of TNF-α. Therefore, the present invention will be significantly more effective in treating the inflammation of gingivitis as compared to current prevention and treatment measures.

In the disclosed composition, the naturally occurring TNF-α modulators comprise resveratrol, caffeic acid, taurine, gallic acid, D-glucosamine, N-acetylcysteine (NAC), salicylic acid, catechol, 3,4-dihydroxybenzoic acid, dopamine, and noradrenaline. These can be used alone or in combination according to the present invention.

Resveratrol is found grapes, peanuts, eucalyptus, spruce etc. Caffeic acid is a naturally occurring compound found in many fruits, vegetables, and herbs, including coffee, although varying in amounts depending on the plant. Taurine is a major constituent of bile and can be found in the lower intestine and in small amounts in the tissues of many animals and in humans as well. Gallic acid is an organic acid found in sumac, witch hazel, tea, oak bark, and other plants. D-Glucosamine is made naturally in the form of glucosamine-6-phosphate, and is the biochemical precursor of all nitrogen-containing sugars. NAC (N-acetylcysteine) is a precursor in the formation of the antioxidant glutathione in the body. Salicylic acid is extracted from willow bark and also isolated from the herb meadowsweet by German researchers in 1839. Catechol is in kino and in beechwood tar. 3,4-dihydroxybenzoic acid is an antioxidant found in roselle. Dopamine is a hormone and neurotransmitter occurring in a wide variety of animals, including both vertebrates and invertebrates. Noradrenaline is a hormone and a neurotransmitter.

The composition may further include one or a plurality of additional beneficial ingredients comprising hexetidine, chlorhexidine digluconate, hydrogen peroxide, ethanol, vitamin B2, calcium, and gum base.

Hexetidine is an anti-bacterial and anti-fungal agent commonly used in both veterinary and human medicine. Chlorhexidine digluconate is an antiseptic that kills both gram-positive and gram-negative microbes. It is also bacteriostatic. Hydrogen peroxide is an antimicrobial agent which can produce oxygen to provide an environment unfavorable for the anaerobic bacteria that grow between the gums and teeth. Ethanol is an antiseptic. Vitamin B2 and calcium help protect against gingivitis as shown by Petti et al. (Community Dentistry & Oral Epidemiology. 28(6):407-13, December 2000). Any of these additional ingredients can provide additional functions of the composition in addition to the modulation of TNF-α. This makes the composition unique and more effective.

In the composition, calcium can be in the form of calcium salt, which can be an inorganic salt, organic salt, or salt formed with amino acid. Examples include calcium carbonate, calcium bicarbonate, calcium chloride, calcium nitrate, calcium oxalate, calcium succinate, calcium acetate, calcium aspartate, and calcium glutamate.

In the composition, the gum base can comprise one or a plurality of elastomer, resin, wax, fat, emulsifier, and talc. The elastomer can comprise loquat and chicle; the resin can comprise polyvinyl acetate and terpene resins; the wax can comprise paraffin and microcrystalline wax; the fat can comprise hydrogenated vegetable oil; the emulsifier can comprise lecithin and glucerol monostearate.

The composition can further comprise one or a plurality of pharmaceutically acceptable excipients comprising water, menthol, sorbitol, paraben and fragrance.

The composition disclosed herein can be orally administered in any convenient form, including as liquid mouthwash or rinse, in a toothpaste composition, as a chewing gum, or a lozenge.

Four examples of the composition are shown below. In the examples, percentages are by weight.

### EXAMPLE 1

### A Mouthwash with the Following Composition

Caffeic acid 5%, N-acetylcysteine 1%, vitamine B2 1%, calcium carbonate 2%, ethanol 20%, water 69.8%, menthol 1%, methyl paraben 0.2%

### EXAMPLE 2

### A Mouthwash with the Following Composition

Resveratrol 2%, gallic acid 2%, vitamin B2 1%, calcium carbonate 2%, water 68%, ethanol 25%, fragrance (trace amount)

### EXAMPLE 3

### A Mouthwash with the Following Composition

Taurine 5%, dopamine 1%, chlorhexidine digluconate 1%, hydrogen peroxide 1%, water 80.8%, ethanol 10%, menthol 1%, ethyl paraben 0.2%

### EXAMPLE 4

### A Chewing Gum with the Following Compositon

Resveratrol 3%, gallic acid 2%, vitamin B2 1%, water 2%, chicle 27%, polyvinyl acetate 15%, hydrogenated vegetable oil 2%, lecithin 3%, fructose 34%, talc 10%, paraffim 1%, fragrance (trace amount)

The present invention also provides a composition for preventing or treating gingivitis and/or maintaining healthy gums wherein the composition comprises one or a plurality of synthetic TNF-α modulators wherein the synthetic TNF-α modulators comprises leflunomide, A77 1726 (teriflunomide), UTL-5b, UTL-5d, and UTL-5g with the structures shown below:

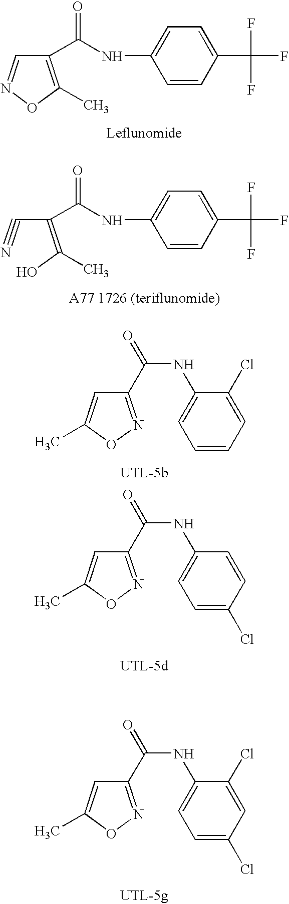

Manna et al. (J. Immunol, 2000;165:5962-9) has shown that leflunomide suppresses TNF-α induced cellular responses. Yao et al. (Inflammation 2004; 28: 97-103) has shown that the metabolite of leflunomide, A 771726, inhibits TNF-α and IL-1 from Kupffer cells. Shaw el al. (British J. of Dermatology 2007; 157: 575-579) has shown the inhibition of TNF-α by a small molecule, UTL-5d.

The composition of the present invention can further comprise vitamin B2 and calcium, wherein said calcium is in the form of calcium salt.

The composition according to the present invention can further comprise one or a plurality of pharmaceutically acceptable excipients comprising water, menthol, sorbitol, paraben and fragrance.

The present invention also discloses a composition for preventing and/or treating gingivitis wherein the composition comprises one or more natural TNF-α modulator and one or more synthetic TNF-αmodulator. The composition can further comprise vitamin B2 and calcium, wherein the calcium is in the form of calcium salt. The composition can further comprise one or a plurality of pharmaceutically acceptable excipients comprising water, menthol, sorbitol, paraben and fragrance.

The present invention also provides a method for preventing and/or treating gingivitis which comprises orally administering the compositions disclosed herein. For example, one method of preventing and/or treating gingivitis according to the present invention can comprise gargling the mouthwash composition described above. Other manners of oral administration can include having a subject use a form of the composition as liquid rinse, in a toothpaste composition, as a chewing gum, or a lozenge, or any manner that exposes the subject's gums to the compositions.

Although the description above contains many specificities, these should not be construed as limiting the scope of the invention but as merely providing the illustrations of some of the presently preferred embodiments of this invention. Thus the scope of this invention should be determined by the appended claims and their legal equivalents, rather than by the examples given.

1. A composition for preventing and/or treating gingivitis wherein the composition comprises one or a plurality of naturally occurring TNF-α modulators.

2. The composition according to claim 1, wherein the naturally occurring TNF-α modulators comprise resveratrol, caffeic acid, taurine, gallic acid, D-glucosamine, N-acetylcysteine (NAC), salicylic acid, catechol, 3,4-dihydroxybenzoic acid, dopamine, and noradrenaline.

3. The composition according to claim 2 further comprising one or a plurality of additional beneficial ingredients comprising hexetidine, chlorhexidine digluconate, hydrogen peroxide, ethanol, vitamin B2, calcium, and gum base.

4. The composition according to claim 3, wherein said calcium is in the form of calcium salt and said gum base comprises one or a plurality of elastomer, resin, wax, fat, emulsifier, and talc.

5. The composition according to claim 4 further comprising one or a plurality of pharmaceutically acceptable excipients comprising water, menthol, sorbitol, paraben and fragrance.

6. The composition according to claim 5, wherein the composition is in the form of liquid mouthwash or rinse, in a toothpaste composition, as a chewing gum, or a lozenge.

7. A composition for preventing or treating gingivitis and/or maintaining healthy gums wherein the composition comprises one or a plurality of synthetic TNF-α modulators.

8. The composition according to claim 7, wherein the synthetic TNF-α modulators comprises leflunomide, A77 1726 (teriflunomide), UTL-5b, UTL-5d, and UTL-5g with the structures shown below:

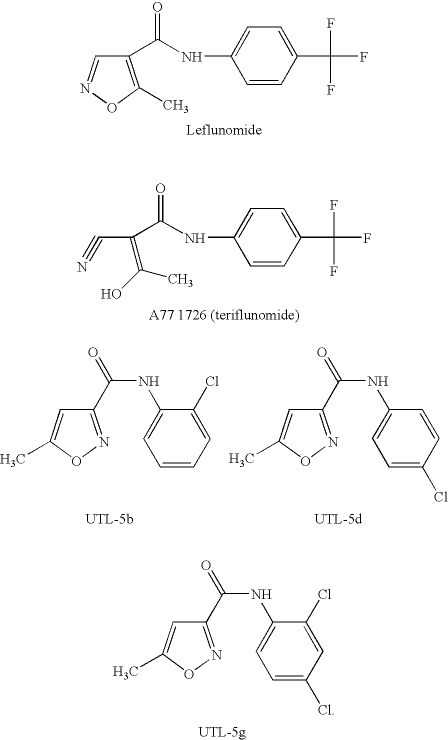

9. The composition in claim 8 further comprising vitamin B2 and calcium.

10. The composition according to claim 8, wherein said calcium is in the form of calcium salt.

11. The composition according to 10 further comprising one or a plurality of pharmaceutically acceptable excipients comprising water, menthol, sorbitol, paraben and fragrance.

12. A method for preventing or treating gingivitis and/or maintaining healthy gums comprising orally administrating the composition in claim 11.

13. A composition for preventing and/or treating gingivitis wherein the composition comprises one natural TNF-α modulator and one synthetic TNF-α modulator.

14. The composition in claim 13 further comprising vitamin B2 and calcium.

15. The composition according to claim 14, wherein said calcium is in the form of calcium salt.

16. The composition according to claim 15 further comprising one or a plurality of pharmaceutically acceptable excipients comprising water, menthol, sorbitol, ethanol, paraben and fragrance.

17. A method for preventing and/or treating gingivitis comprising orally administrating the composition in claim 16.
